(19) United States
(12) Patent Application Publication
(10) Pub. No.: US 2025/0032268 A1
(43) Pub. Date: Jan. 30, 2025

(54) Anatomical Talar Component Design for Total Ankle Replacement

(71) Applicant: Arthrex, Inc., Naples, FL (US)

(72) Inventors: Adam N. Garlock, Naples, FL (US); Braham Dhillon, Naples, FL (US); Maris Prieditis, Naples, FL (US); Benjamin Chan, Bonita Springs, FL (US); Brian Dorn, Naples, FL (US); Andrea Matuska, Naples, FL (US)

(21) Appl. No.: 18/714,218

(22) PCT Filed: Dec. 2, 2022
(86) PCT No.: PCT/US2022/051611

§ 371 Date: May 29, 2024

Related U.S. Application Data
(60) Provisional application No. 63/285,690, filed on Dec. 3, 2021.
(60) Provisional application No. 63/337,556, filed on May 2, 2022.

(51) Int. Cl. A61F 2/42; A61F 2/30

(52) U.S. Cl. CPC A61F 2/4202; A61F 2002/30327; A61F 2002/30593; A61F 2002/30825; A61F 2002/3092; A61F 2002/30985; A61F 2002/4205; A61F 2002/4207; A61F 2310/00017; A61F 2310/00023; A61F 2310/00029; A61F 2310/00083

(57) ABSTRACT

The present disclosure provides a prosthetic ankle including a talar component having a top surface and a bottom surface. The bottom surface is configured to be positioned adjacent to a talus. The top surface includes a trochlear groove extending from a posterior side of the talar component to an anterior side of the talar component. The trochlear groove includes a first portion adjacent the posterior side of the talar component and a second portion adjacent the anterior side of the talar component.

### CROSS-REFERENCE TO RELATED APPLICATIONS

This application claims the benefit of priority to (i) U.S. Provisional Application No. 63/285,690 entitled “Anatomical Talar Component Design for Total Ankle Replacement,” filed on Dec. 3, 2021, and (ii) U.S. Provisional Application No. 63/337,556 entitled “Prosthetic Implant with a Zinc-Strontium Alloy for Stem Cell Stimulation,” filed on May 2, 2022, the contents of each of which are hereby incorporated by reference in their entirety.

### BACKGROUND

A talar implant with an anatomic trochlear surface with a multiaxial axis of rotation allows a total ankle replacement prosthetic to mimic the natural kinematics during gait. Anatomic talar implants are disclosed herein.

### SUMMARY

The disclosure herein includes a talar component for a prosthetic ankle. The talar component described herein allows for mobility similar to the native ankle joint by allowing for coupled motion during flexion and extension.

In particular, a prosthetic ankle is designed to replicate the natural kinematics of the ankle. The rotational axis of the talar component of the prosthetic ankle is skewed and oriented as a compound angle in transverse and coronal planes. In addition to skewing the rotational axis of the talar component, the talar component is designed with varying radii with a larger medial radius and a smaller lateral radius which also aids in replicating the natural joint function. To aid in the ankles increased ability to internally and externally rotate during dorsiflexion, a tapered trochlear groove that widens posteriorly allows for this motion while maintaining stability in neutral and dorsiflexion stances. A trochlear groove design as disclosed herein can aid the ankle in maintaining medial/lateral stability.

In addition, to reduce weight while providing strength, a talar component can include an internal lattice structure to provide the strength required to oppose ground reaction forces during normal gait. The bottom surface of the talar component may be coupled to a bone interfacing porous structure to promote bone ingrowth/on growth and may include one or more channels that will allow the user to insert cement and biologics with a proper delivery system after implantation. These channels are located between the solid body of the talar component and the porous bone interface surface.

Thus, in one aspect, a prosthetic ankle can include a talar component having a top surface and a bottom surface. The bottom surface is configured to be positioned adjacent to a talus. The top surface includes a trochlear groove extending from a posterior side of the talar component to an anterior side of the talar component. The trochlear groove includes a first portion adjacent the posterior side of the talar component and a second portion adjacent the anterior side of the talar component.

As discussed above, prosthetic implants are used in a variety of medical procedures. In such procedures, at least part of the prosthetic implant may be inserted into a bone of the patient. A common failure mode in such procedures is the loosening or subsidence of the prosthetic implant after implantation. Osseointegration of the patient's anatomy and the prosthetic implant surface is a critical step in the healing process and may contribute to the longevity and success of the prosthetic implant by reducing the likelihood of implant loosening.

As such, the disclosure herein further includes a prosthetic implant with a Zinc-Strontium (Zn—Sr) interface surface to aid in the stimulation of osteogensis and osseointegration at the implant site.

Current prosthetic implants, such as talar and tibial components for total ankle replacement (TAR) as non-limiting examples, are generally coated with Ti Plasma spray, hydroxyapatite (HA), or calcium phosphate (CaP). Recent advancements in additive manufacturing have led to porous or scaffold like structures being used in lieu of Ti Plasma spray to further promote bone ingrowth with the implant. Although these bone ingrowth surfaces provide the potential for osseointegration they lack osteogenic activity and thus do not promote new bone formation. Adding Zn—Sr based metals to the porous ingrowth surface of various prosthetic implants would not only aid in the stimulation of osteogenesis and osseointegration, but this increased response could reduce healing time after surgery while providing increased construct fixation. The increased ossification response of Zn—Sr alloys could potentially reduce the likelihood of implant loosening or subsidence given that they inhibit bone resorption while promoting new bone formation.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a top view of an example talar component of a prosthetic ankle.

FIG. 2 is a front view of the example talar component of FIG. 1.

FIG. 3 is a front view of the example talar component of FIG. 1 illustrating the varying medial and lateral radii of the top surface.

FIG. 4 is a side view of the example talar component of FIG. 1.

FIG. 5 is side cross-sectional view of the example talar component of FIG. 1 illustrating a lattice structure.

FIG. 6 is a bottom view of an example talar component illustrating a porous structure.

FIG. 7 is a bottom view of an example talar component illustrating one or more channels.

FIG. 8 is a bottom view of another example talar component illustrating a shell structure with an internal lattice structure.

FIG. 9 is a bottom view of another example talar component illustrating a lattice structure positioned within the shell of FIG. 8.

### DETAILED DESCRIPTION

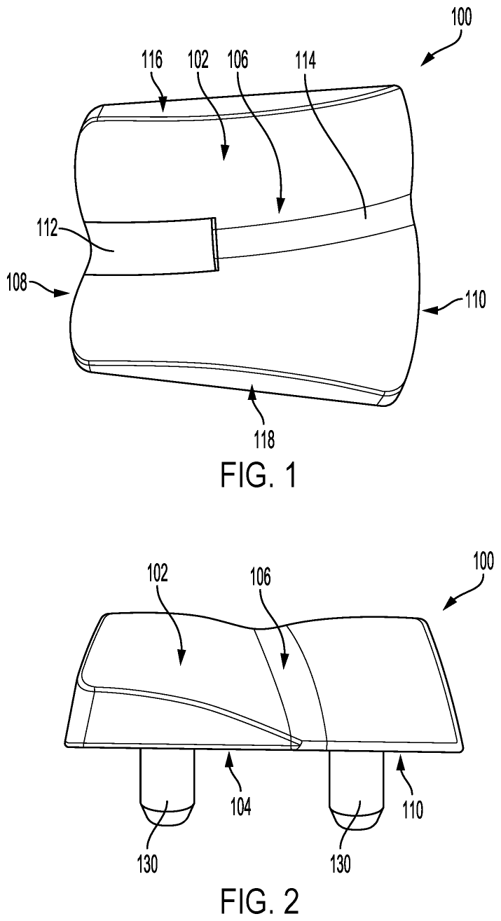

With reference to the Figures, FIGS. 1-2 illustrate a talar component 100 of a prosthetic ankle. The talar component 100 includes a top surface 102 and a bottom surface 104 opposite the top surface 102. The bottom surface 104 is configured to be positioned adjacent to a talus of a patient. The top surface 102 includes a trochlear groove 106 extending from a posterior side 108 of the talar component 100 to an anterior side 110 of the talar component 100. The trochlear groove 106 includes a first portion 112 adjacent the posterior side 108 of the talar component 100 and a second portion 114 adjacent the anterior side 110 of the talar component 100. In an example, a diameter of the first portion 112 of the trochlear groove 106 is different from a diameter of the second portion 114 of the trochlear groove 106. In an example, the diameter of the first portion 112 of the trochlear groove 106 is greater than the diameter of the second portion 114 of the trochlear groove 106. In another example, the diameter of the first portion 112 of the trochlear groove 106 is less than the diameter of the second portion 114 of the trochlear groove 106

In an example, as shown in FIG. 1, the trochlear groove 106 is skewed in a lateral direction as the trochlear groove 106 extends from the posterior side 108 of the talar component 100 to the anterior side 110 of the talar component 100. As shown in FIGS. 1-2, in an example the top surface 102 of the talar component 100 is skewed superiorly from a medial direction to a lateral direction, and the top surface 102 of the talar component 100 is skewed posteriorly from an anterior direction to a posterior direction. As such, the rotational axis of the talar component of the prosthetic ankle is skewed and oriented as a compound angle in transverse and coronal planes. In an example, the axis of revolution of the talar component 100 is skewed superiorly from medial to lateral in the coronal plane and is skewed posteriorly from medial to lateral in the transverse plane. In an example, the axis of rotation is skewed in the same direction as the top surface 102.

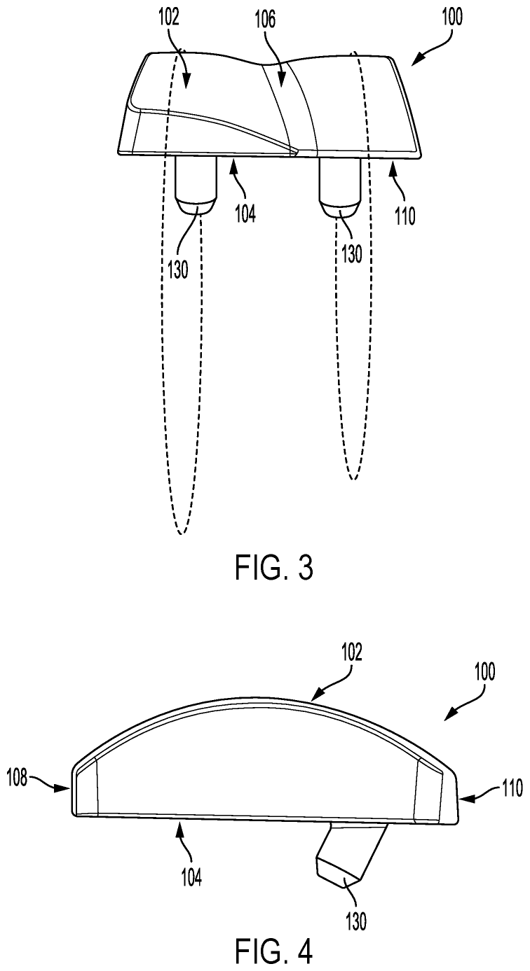

In an example, the talar component 100 is designed with varying radii with a larger medial radius and a smaller lateral radius. In particular, as shown in FIG. 3, an average radius of curvature of a medial portion of the top surface 102 of the talar component 100 is greater than an average radius of curvature of a lateral portion of the top surface 102 of the talar component 100. In one such example, the medial portion comprises a portion of the top surface 102 of the talar component 100 that is medial to the trochlear groove 106, and the lateral portion comprises a portion of the top surface 102 of the talar component 100 that is lateral to the trochlear groove 106. The skewed axis of rotation and the varying radii of the talar component 100 described above aids in replicating the natural joint kinematics of the ankle which allows for mobility similar to the native ankle joint by allowing for internal rotation and inversion as the ankle moves into plantarflexion and external rotation and eversion as the ankle moves into dorsiflexion.

Further, as shown in FIG. 2, a height of the medial portion of the top surface 102 at the anterior side 110 of the talar component 100 is variable, while a height of the lateral portion of the top surface 102 at the anterior side 110 of the talar component 100 is constant. In an example, the height of the lateral portion of the top surface 102 at the anterior side 110 of the talar component 100 is zero.

In an example, the diameter of the first portion 112 of the trochlear groove 106 (in the coronal plane) can be about 10 mm to about 16 mm, and the diameter of the second portion 114 of the trochlear groove 106 (in the medial lateral direction) can be about 8 mm to about 12 mm. The diameter of the first portion 112 of the trochlear groove 106 (in the medial lateral direction) can be about 10 mm, about 11 mm, about 12 mm, about 13 mm, about 14 mm, about 15 mm, or about 16 mm. The diameter of the second portion 114 of the trochlear groove 106 (in the coronal plane) can be about 8 mm, about 9 mm, about 10 mm, about 11 mm, or about 12 mm. In an example, the diameter of the first portion 112 of the trochlear groove 106 is constant along its length, and the diameter of the second portion 114 of the trochlear groove 106 is also constant along its length. In another example, the diameter of the first portion 112 of the trochlear groove 106 is variable along its length, and the diameter of the second portion 114 of the trochlear groove 106 is also variable along its length. In another example, the diameter of the first portion 112 of the trochlear groove 106 is variable along its length, and the diameter of the second portion 114 of the trochlear groove 106 is constant along its length. In yet another example, the diameter of the first portion 112 of the trochlear groove 106 is constant along its length, and the diameter of the second portion 114 of the trochlear groove 106 is variable along its length. As such, the trochlear groove 106 can have varying radii from anterior to posterior (the radius of curvature that the trochlear groove 106 follows can have a first radius anteriorly and a second radius posteriorly) while having either a constant or varying medial-lateral diameter (width of groove).

In an example, the first portion 112 of the trochlear groove 106 has a first length, the second portion 114 of the trochlear groove 106 has a second length, and the second length is greater than the first length. The tapered trochlear groove 106 that widens posteriorly as described above aids in the ankles increased ability to internally and externally rotate during plantarflexion and dorsiflexion while maintaining stability in neutral and dorsiflexion stances.

In an example, as shown in FIG. 1, the posterior side 108 of the talar component 100 has a first width, the anterior side 110 of the talar component 100 has a second width, and the second width is greater than the first width. The talar component 100 may further include a first sidewall 116 positioned between the top surface 102 and the bottom surface 104 of the talar component 100, and a second sidewall 118 positioned between the top surface 102 and the bottom surface 104 of the talar component 100. In one such example, a minimum width of the bottom surface 104 is greater than a minimum width of the top surface 102 such that each of the first sidewall 116 and the second sidewall 118 are angled inwards from the bottom surface 104 towards the top surface 102.

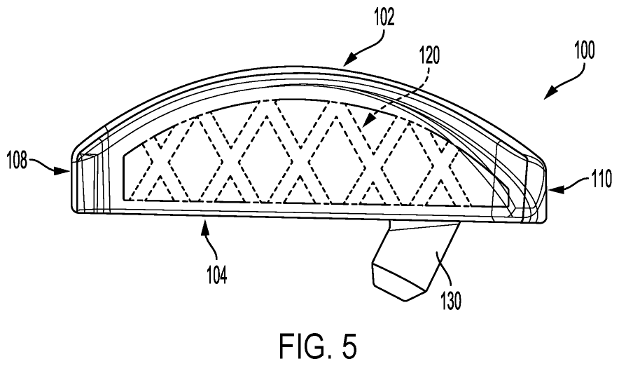

Most modern arthroplasty devices that articulate with a bearing surface are manufactured from cobalt-chromium (CoCr) alloys, ceramic alloys, oxidized Zirconium, and Nitride coated Titanium alloys for improved wear resistance. However, CoCr and the previously mentioned materials are dense materials, whose increased weight can cause increased wear against the less dense bone that the implant resides upon. In order to minimize such wear and reduce the weight of the implant, while preserving the desirable properties of CoCr, weight reducing mechanisms are desirable. In an example, as shown in FIG. 5, an interior of the talar component 100 is hollow. In one such example, the interior of the talar component 100 includes a lattice structure 120. In an example, an entirety of the interior of the talar component 100 comprises the lattice structure 120. In another example, the interior of the talar component 100 includes alternating solid layers and lattice structure layers. The solid and lattice layers can be manufactured from the same material (such as CoCr) or a variation of mixed material layers. This same material may also comprise the shell of the talar component 100 as well. The lattice structure 120 positioned in the hollow interior of the talar component 100 that adds strength to the implant can be either be a uniform beam design or a formula driven gyroid shape. The porous structure 122 can be created and defined as a stochastic type for the on-growth/ingrowth surface. Although the material can be the same, the cell type and structure of both the lattice structure 120 and porous structure 122 features can be different.

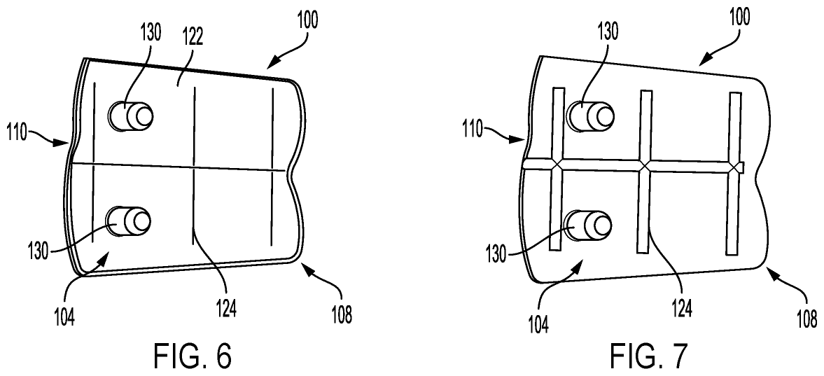

In an example, as shown in FIG. 6, the talar component 100 includes a porous structure 122 positioned adjacent the bottom surface 104. In an example, the porous structure 122 may include separate surfaces or structures that are sintered, diffusion bonded, or additively manufactured to the bottom surface 104. The porous structure 122 may advantageously promote bone ingrowth/on growth of the talar component 100. In another example, as shown in FIG. 7, the talar component 100 includes one or more channels 124 embedded in the porous structure 122. The one or more channels 124 may be interconnected so that they are each in fluid communication with one another. In an example, at least one channel of the one or more channels 124 extends to the anterior side 110 of the talar component 100. As such, one channel of the one or more channels 124 that extends to the anterior side 110 of the talar component 100 provides access to a user to inject a bone cement and/or other biologics into the porous structure 122 to further promote bone ingrowth/on growth of the talar component 100.

In an example, the one or more channels 124 comprise a first channel extending in a direction from the posterior side 108 of the talar component 100 to the anterior side 110 of the talar component, the one or more channels 124 further comprise a second channel extending in a lateral direction from the first channel, and the one or more channels 124 further comprise a third channel extending in a medial direction from the first channel.

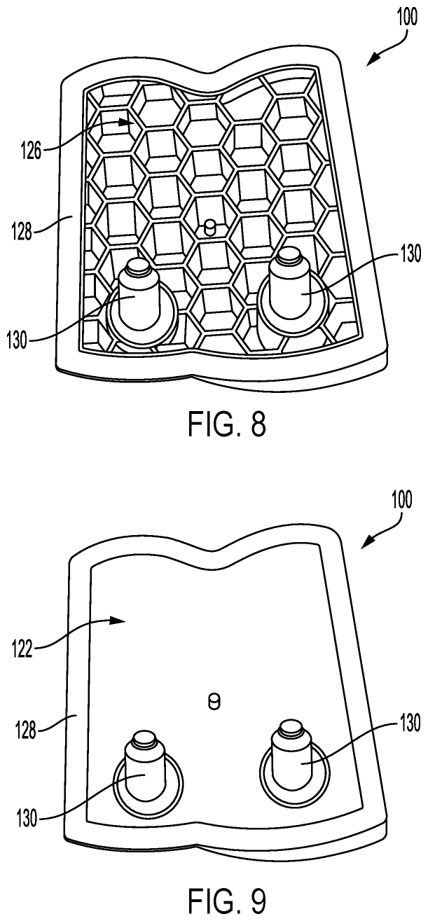

FIG. 8 illustrates a shell 126 of the talar component 100, while FIG. 9 illustrates the lattice structure 120 positioned throughout the hollow portions of the shell 126. The porous structure 122 (not shown in FIGS. 8 and 9) would cover both the lattice structure 120 and the edges 128 of the shell 126 of the bottom surface 104 of the talar component 100.

As further shown in FIGS. 8-9, the bottom surface 104 includes one or more interosseous fixation elements 130 extending away from the bottom surface 104. In an example, one or more interosseous fixation elements 130 comprise a pair of talar pegs. In use, the one or more interosseous fixation elements 130 are configured to be positioned within the talus of the patient. In an example, an interior of each of the one or more interosseous fixation elements 130 are solid. In another example, an interior of each of the one or more interosseous fixation elements 130 are hollow and include a lattice structure similar to the lattice structure 120 of the main body of the talar component 100 discussed above. In an example, each of the one or more interosseous fixation elements 130 further include one or more channels for injecting bone cement and/or other biologics therein. In an example, each of the one or more interosseous fixation elements 130 further include a porous structure positioned on an exterior of the one or more interosseous fixation elements 130 to thereby promote bone ingrowth/on growth one or more similar to the porous structure 122 of the bottom surface 104 of the talar component 100 discussed above. In an example, each of the one or more interosseous fixation elements 130 are angled between 0 and 90 degrees with respect to the bottom surface 104 of the talar component 100. In another example, each of the one or more interosseous fixation elements 130 are perpendicular to the bottom surface 104 of the talar component 100.

The talar component 100 described herein allows for mobility similar to the native ankle joint by allowing for coupled motion during flexion and extension. The talar component 100 allows for internal rotation and inversion as the ankle moves into plantarflexion and external rotation and eversion as the ankle moves into dorsiflexion.

The talar component 100 may further include a bearing surface and a tibial component having a top surface configured to be positioned adjacent to a tibia and a bottom surface configured to be positioned adjacent a top surface of the bearing surface. In an example, the bearing surface comprises ultra-high-molecular-weight polyethylene (UHMWPE). In an example, a bottom surface of the bearing surface is configured to substantially match the top surface 102 of the talar component 100 such that the bearing surface and tibial component can move relative to one another and frictionally engage one another on the top surface of the bearing surface. In an example, the bottom surface of the bearing surface is configured for at least partially constraining a mobility of the bearing surface relative to the tibial component.

In an example, the bottom surface 104 of the talar component 100 is configured to be positioned in contact with a bone of a patient, and at least a portion of an exterior surface of the bottom surface 104 includes a Zinc-Strontium (Zn—Sr) alloy. In such an example, the Zn—Sr alloy is selected from the group consisting of Zn—Sr, Zn-0.8Sr, Zn-0.6 Sr, Zn-0.SSr, Zn-0.4Sr, Zn-0.2Sr, and Zn-0.1 Sr. Once the second end of the prosthetic implant is in contact with the bone of the patient, the Zn—Sr alloy stimulates osteogeneis of mesenchymal stem cells at the implant site. In an example, the Zn—Sr alloy stimulates mesenchymal stem cells selected from the group consisting of CD45−, CD45′/CD146+, CD45−CD271+, CD31−44+45−73+90+105+, and CD45−CD34+. Further, the Zn—Sr alloy increases cellular PI3K/Akt, MAPK/Erk, and/or Wnt/β-catenin pathway signaling, thereby promoting anabolic and anticatabolic effects on bone remodeling. In an example, the Zn—Sr alloy further includes a material selected from the group consisting of tricalcium phosphate (TCP), hydroxyapatite (HA), and Silicon. In another example, the Zn—Sr alloy includes no more than a trace amount of Magnesium.

The addition of Sr—Zn based metals to the bottom surface 104 of the talar component 100 for total ankle replacement surgeries would help promote and/or stimulate new bone formation while also inhibiting bone resorption during the healing process thereby reducing potential failure modes associated with implant loosening and subsidence.

In an example, the top surface 102 of the talar component 100 is configured to extend away from the bone after implantation of the prosthetic implant in the bone. In one such example, an exterior surface of the top surface 102 of the talar component 100 comprises a first material, and the exterior surface of the bottom surface 104 of the talar component 100 comprises a second material that is different from the first material. In one such example, the first material comprises a titanium alloy, stainless steel, polyetheretherketone (PEEK), or a cobalt-chromium (CoCr) alloy, and the second material comprises the Zn—Sr alloy.

In an example, the Zn—Sr alloy comprises a three-dimensional structure extending away from the exterior surface of the bottom surface 104. In one such example, the three-dimensional structure comprises a scaffold.

In some examples, or more components of the prosthetic implant is made via an additive manufacturing process using an additive-manufacturing machine, such as stereolithography, multi-jet modeling, inkjet printing, selective laser sintering/melting (or DMLS, EBM), and fused filament fabrication, among other possibilities. Additive manufacturing enables one or more components of the prosthetic implant and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the prosthetic implant using a Computer Aided Design (CAD) of the prosthetic implant as instructions. As a result, changes to the design of the prosthetic implant can be immediately carried out in subsequent physical creations of the prosthetic implant. This enables the components of the prosthetic implant to be easily adjusted or scaled to fit different types of applications (e.g., for use with various types and sizes of patient anatomy).

The layer-upon-layer process utilized in additive manufacturing can deposit one or more components of the prosthetic implant with complex designs that might not be possible for devices assembled with subtractive manufacturing. In turn, the design of the prosthetic implant can include aspects that aim to improve overall operation. For example, the design can incorporate physical elements that help redirect stresses in a desired manner that traditionally manufactured devices might not be able to replicate.

Additive manufacturing also enables depositing one or more components of the prosthetic implant in a variety of materials using a multi-material additive-manufacturing process. In such an example, the exterior surface of the first end of the prosthetic implant may be made from a first material, and the exterior surface of second end of the prosthetic implant may be made from a second material that is different than the first material. In another example, the entire prosthetic implant is made from the same material. Other example material combinations are possible as well. Further, one or more components of the prosthetic implant can have some layers that are created using a first type of material and other layers that are created using a second type of material.

In an example, an interior of one or more components the prosthetic implant is hollow. In one such example, the interior of the prosthetic implant includes a lattice structure. In an example, an entirety of the interior of the prosthetic implant comprises the lattice structure. In another example, the interior of the prosthetic implant includes alternating solid layers and lattice structure layers. The solid and lattice layers can be manufactured from the same material (such as CoCr) or a variation of mixed material layers. This same material may also comprise the shell of prosthetic implant as well. The lattice structure positioned in the hollow interior of the prosthetic implant that adds strength to the implant can be either be a uniform beam design or a formula driven gyroid shape.

In some examples, such as shown in any one of FIGS. 1-9, one or more components of the talar component 100 is made via an additive manufacturing process using an additive-manufacturing machine, such as stereolithography, multi-jet modeling, inkjet printing, selective laser sintering/melting, and fused filament fabrication, among other possibilities. Additive manufacturing enables one or more components of the talar component 100 and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the talar component 100 using a Computer Aided Design (CAD) of the talar component 100 as instructions. As a result, changes to the design of the talar component 100 can be immediately carried out in subsequent physical creations of the talar component 100. This enables the components of the talar component 100 to be easily adjusted or scaled to fit different types of applications (e.g., for use with various types and sizes of prosthetic ankles).

The layer-upon-layer process utilized in additive manufacturing can deposit one or more components of the talar component 100 with complex designs that might not be possible for devices assembled with subtractive manufacturing. In turn, the design of the talar component 100 can include aspects that aim to improve overall operation. For example, the design can incorporate physical elements that help redirect stresses in a desired manner that traditionally manufactured devices might not be able to replicate.

Additive manufacturing also enables depositing one or more components of the talar component 100 in a variety of materials using a multi-material additive-manufacturing process. In such an example, the majority of the talar component 100 may be made from a first material and lattice structure 120 and/or the porous structure 122 may be made from a second material that is different than the first material. In another example, the entire talar component 100 is made from the same material. Other example material combinations are possible as well. Further, one or more components of the talar component 100 can have some layers that are created using a first type of material and other layers that are created using a second type of material.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” or “an example” means that one or more feature, structure, or characteristic described in connection with the example is included in at least one implementation. The phrases “one embodiment” or “one example” or “an example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ±5% of a given value.

Illustrative, non-exhaustive examples, which may or may not be claimed, of the subject matter according the present disclosure are provided below.

What is claimed is:

1. A prosthetic ankle comprising:
a talar component having a top surface and a bottom surface, wherein the bottom surface is configured to be positioned adjacent to a talus, wherein the top surface includes a trochlear groove extending from a posterior side of the talar component to an anterior side of the talar component, wherein the trochlear groove includes a first portion adjacent the posterior side of the talar component and a second portion adjacent the anterior side of the talar component.

2. The prosthetic ankle of claim 1, wherein the trochlear groove is skewed in a lateral direction as the trochlear groove extends from the posterior side of the talar component to the anterior side of the talar component.

3. The prosthetic ankle of any one of claims 1-2, wherein the top surface of the talar component is skewed superiorly from a medial direction to a lateral direction, and wherein the top surface of the talar component is skewed posteriorly from an anterior direction to a posterior direction.

4. The prosthetic ankle of any one of claims 1-3, wherein an average radius of curvature of a medial portion of the top surface of the talar component is greater than an average radius of curvature of a lateral portion of the top surface of the talar component.

5. The prosthetic ankle of claim 4, wherein the medial portion comprises a portion of the top surface of the talar component that is medial to the trochlear groove, and wherein the lateral portion comprises a portion of the top surface of the talar component that is lateral to the trochlear groove.

6. The prosthetic ankle of any one of claims 1-5, wherein a diameter of the first portion of the trochlear groove is different from a diameter of the second portion of the trochlear groove.

7. The prosthetic ankle of claim 6, wherein the diameter of the first portion of the trochlear groove is greater than the diameter of the second portion of the trochlear groove.

8. The prosthetic ankle of claim 6, wherein the diameter of the first portion of the trochlear groove is greater than the diameter of the second portion of the trochlear groove.

9. The prosthetic ankle of any one of claims 6-8, wherein the diameter of the first portion of the trochlear groove ranges from about 10 mm to about 16 mm, and wherein the diameter of the second portion of the trochlear groove ranges from about 8 mm to about 12 mm.

10. The prosthetic ankle of any one of claims 6-9, wherein the diameter of the first portion of the trochlear groove is constant, and wherein the diameter of the second portion of the trochlear groove is constant.

11. The prosthetic ankle of any one of claims 6-9, the diameter of the first portion of the trochlear groove is variable, and wherein the diameter of the second portion of the trochlear groove is variable.

12. The prosthetic ankle of any one of claims 6-11, wherein the first portion of the trochlear groove has a first length, wherein the second portion of the trochlear groove has a second length, and wherein the second length is greater than the first length.

13. The prosthetic ankle of any one of claims 1-12, wherein the posterior side of the talar component has a first width, wherein the anterior side of the talar component has a second width, and wherein the second width is greater than the first width.

14. The prosthetic ankle of any one of claims 1-13, further comprising:
a first sidewall positioned between the top surface and the bottom surface of the talar component; and
a second sidewall positioned between the top surface and the bottom surface of the talar component, wherein a minimum width of the bottom surface is greater than a minimum width of the top surface such that each of the first sidewall and the second sidewall are angled inwards from the bottom surface towards the top surface.

15. The prosthetic ankle of any one of claims 1-14, wherein an interior of the talar component is hollow.

16. The prosthetic ankle of claim 15, wherein the interior of the talar component includes a lattice structure.

17. The prosthetic ankle of claim 15, wherein the interior of the talar component includes alternating solid layers and lattice structure layers.

18. The prosthetic ankle of any one of claims 1-17, wherein talar component includes a porous structure positioned adjacent the bottom surface.

19. The prosthetic ankle of claim 18, wherein talar component includes one or more channels embedded in the porous structure.

20. The prosthetic ankle of claim 19, wherein the one or more channels are interconnected so that they are each in fluid communication with one another.

21. The prosthetic ankle of any one of claims 19-20, wherein at least one channel of the one or more channels extends to the anterior side of the talar component.

22. The prosthetic ankle of any one of claims 1-21, wherein the bottom surface includes one or more interosseous fixation elements extending away from the bottom surface, and wherein the one or more interosseous fixation elements are configured to be positioned within the talus.

23. The prosthetic ankle of claim 22, wherein an interior of each of the interosseous fixation elements are hollow and include a lattice structure.

24. The prosthetic ankle of any one of claims 22-23, wherein the one or more interosseous fixation elements comprise a pair of talar pegs, and wherein each of the pair of talar pegs are angled between 0 and 90 degrees with respect to the bottom surface of the talar component.

25. The prosthetic ankle of claim 24, wherein each of the pair of talar pegs are perpendicular to the bottom surface of the talar component.

26. The prosthetic ankle of any one of claims 1-25, further comprising:
a bearing surface; and
a tibial component having a top surface configured to be positioned adjacent to a tibia and a bottom surface configured to be positioned adjacent a top surface of the bearing surface.

27. The prosthetic ankle of claim 26, wherein a bottom surface of the bearing surface is configured to substantially match the top surface of the talar component such that the bearing surface and tibial component can move relative to one another and frictionally engage one another on the top surface of the bearing surface.

28. The prosthetic ankle of any one of claims 1-27, wherein the bottom surface of the talar component is configured to be positioned within a bone of a patient, and wherein at least a portion of an exterior surface of the bottom surface includes a Zinc-Strontium (Zn—Sr) alloy.

29. The prosthetic ankle of claim 28, wherein the Zn—Sr alloy is selected from the group consisting of Zn—Sr, Zn-0.8Sr, Zn-0.6 Sr, Zn-0.SSr, Zn-0.4Sr, Zn-0.2Sr, and Zn-0.1 Sr.

30. The prosthetic ankle of any one of claims 28-29, wherein the Zn—Sr alloy stimulates osteogeneis of mesenchymal stem cells selected from the group consisting of CD45−, CD45′/CD146+, CD45−CD271+, CD31−44+45−73+90+105+, and CD45−CD34+.

31. The prosthetic ankle of any one of claims 28-30, wherein the Zn—Sr alloy increases cellular PI3K/Akt, MAPK/Erk, and/or Wnt/β-catenin pathway signaling, thereby promoting anabolic and anticatabolic effects on bone remodeling.

32. The prosthetic ankle of any one of claims 28-31, wherein the Zn—Sr alloy further includes a material selected from the group consisting of tricalcium phosphate (TCP), hydroxyapatite (HA), and Silicon.

33. The prosthetic ankle of any one of claims 28-32, wherein the Zn—Sr alloy includes no more than a trace amount of Magnesium.

34. The prosthetic ankle of any one of claims 28-33, wherein the top surface of the talar component is configured to extend away from the bone after implantation of the talar component in the bone, wherein an exterior surface of the top surface of the talar component comprises a first material, and wherein the exterior surface of the bottom surface of the talar component comprises a second material that is different from the first material.

35. The prosthetic ankle of claim 34, wherein the first material comprises a titanium alloy, stainless steel, polyetheretherketone (PEEK), or a cobalt-chromium (CoCr) alloy, and wherein the second material comprises the Zn—Sr alloy.

36. The prosthetic ankle of any one of claims 28-35, wherein the Zn—Sr alloy comprises a three-dimensional structure extending away from the exterior surface of the second end.

37. The prosthetic ankle of claim 36, wherein the three-dimensional structure comprises a scaffold.
